(19) United States
(12) Patent Application Publication
(10) Pub. No.: US 2018/0103891 A1
(43) Pub. Date: Apr. 19, 2018

(54) PORTABLE SKIN CONDITION MEASURING DEVICE, SKIN CONDITION ANALYZING METHOD, AND PROGRAM USING THE SAME

(71) Applicant: WAYWEARABLE, INC., Seongnam-si (KR)

(72) Inventors: Jong Soo MOON, Seongnam-si (KR); Min Suk KIM, Seoul (KR)

(73) Assignee: WAYWEARABLE, INC., Seongnam-si (KR)

(21) Appl. No.: 15/838,452

(22) Filed: Dec. 12, 2017

Related U.S. Application Data
(63) Continuation of application No. PCT/KR2016/006554, filed on Jun. 21, 2016.

(30) Foreign Application Priority Data
Jun. 24, 2015 (KR) 10-2015-0089835

(51) Int. Cl. A61B 5/00; A61B 5/053

(52) U.S. Cl. CPC A61B 5/443; A61B 5/0531; A61B 5/0537; A61B 2560/0431; A61B 5/7282; A61B 5/486; A61B 2560/0252; A61B 5/7278

(57) ABSTRACT

Disclosed are a portable skin condition measuring device, a skin condition analyzing method, and a program using the same. The skin condition analyzing method includes receiving input alternating current data and return alternating current data from the skin condition measuring device through a wireless communication, and comparing and analyzing the input alternating current data with the return alternating current data to provide skin condition information. The user identifies the skin condition of the desired skin area at the certain time point in real time. Accordingly, the user instantly checks and performs the appropriate prescription corresponding to the skin condition, and thus the user effectively maintains or improves the skin condition.

### CROSS-REFERENCE TO RELATED APPLICATIONS

The present application is a continuation of International Patent Application No. PCT/KR2016/006554, filed on Jun. 21, 2016, which is based upon and claims the benefit of priority to Korean Patent Application No. 10-2015-0089835, filed on Jun. 24, 2015. The disclosures of the above-listed applications are hereby incorporated by reference herein in their entirety.

### BACKGROUND

Embodiments of the inventive concept described herein relate to a portable skin condition measuring device, a skin condition analyzing method, and a program using the same, and more particularly, relate to a portable skin condition measuring device that applies an alternating current to a user's skin to obtain data for measuring a skin condition, and a method and a program, which analyze the skin condition of the user using the portable skin condition measuring device or a portable terminal connected with the portable skin condition measuring device.

It is not easy for ordinary people to visit esthetics regularly to get skin care treatments due to time and financial restraints even though they pay attention to skin and beauty treatments. In addition, since interest in taking good care of the way they look is increasing among people of all ages, an esthetics-related business, e.g., books or internet sites related to skin and beauty cares, is experiencing an “explosion of growth”. Therefore, most of the people who are not able to visit estheticians find skin care tips and advices from the books or the internet sites according to their skin conditions.

However, people have different skin conditions, and they are unsure of what exact skin conditions they have. Accordingly, they are not able to collect the information to suit their skin needs when using the books or the internet sites.

In addition, a user has to possess the books or login the internet sites to search for the information he or she needs although the user knows his/her skin type and skin condition. Further, skin conditions of people vary depending on surrounding environments, such as temperature, humidity, ultraviolet index, etc., and lengths of outdoor activity time. Thus, a system that enables the user to measure his/her skin condition at a certain time point and easily provides the tips and advices appropriate to the situation in real time is necessary.

Also, when the user wants to purchase cosmetics, the cosmetics store finds it difficult to recommend the cosmetics appropriate for the users' skin types. In recent, a lot of functional cosmetics have been sold with a growing interest in functional cosmetics for healthy restoration and maintenance of the user's skin. The functional cosmetics are products that aid to improve or maintain the skin condition of the user, and numerous types of functional cosmetics are available and becoming increasingly specialized. For the proper use of the functional cosmetics, the accurate measurement and analysis of the skin condition of the user is required to choose the most suitable cosmetics for the skin condition of the user. However, in case of a conventional method of analyzing skin condition, it takes a lot of time, i.e., from a few days to tens of days, to measure, analyze, and evaluate the skin condition, and thus there have been disadvantages in that the customers' needs are not answered immediately. Also, since a deviation in measurement result and analysis result is high depending on the measurement condition and the proficiency of the measurers, the use of the conventional method of analyzing skin condition is extremely low except for some laboratories or skin analysis centers where there are specialized estheticians. Although the cosmetics stores want to immediately respond the customers' requests, it is not easy for the cosmetics stores to have an expensive skin analysis system and to hire a specialized esthetician.

### SUMMARY

Embodiments of the inventive concept provide a portable skin condition measuring device capable of allowing a user to conveniently measure, in real time, a condition of skin of a desired skin area at a desired time point using an alternating current applied to the skin.

In addition, embodiments of the inventive concept provide a skin condition analyzing method and a program using the skin condition analyzing method, which are capable of providing an adequate diagnosis of the user's skin condition by reducing the impact of errors based on input alternating current data and return alternating current data and providing appropriate prescription data according to the diagnosis.

According to an aspect of an embodiment, a method for analyzing a skin condition includes receiving input alternating current data and return alternating current data and comparing the input alternating current data with the return alternating current data to calculate skin condition information.

When the input alternating current data and the return alternating current data respectively correspond to voltage data and the skin condition information is a skin moisture degree, the calculating of the skin condition information includes comparing the voltage data of the input alternating current data with the voltage data of the return alternating current data to calculate voltage drop data, calculating an equivalent impedance based on the calculated voltage drop data, calculating an electrical conductivity based on the equivalent impedance, and calculating the skin moisture degree corresponding to the electrical conductivity.

The receiving of the input alternating current data and the return alternating current data includes receiving the input alternating current data having a plurality of different frequencies and the return alternating current data corresponding to the input alternating current data, the calculating of the skin condition information reflects the skin condition information by frequency, which is calculated depending on each of the frequencies, to calculate accumulated skin condition information, and the skin condition information by frequency corresponds to skin condition information obtained by alternating currents that have different frequencies from each other and move along different paths from each other in a skin layer due to a cell permeability by frequency.

The method further includes calculating a skin oiliness degree corresponding to the calculated skin moisture degree.

The method further includes determining whether the skin condition information exceeds a normal range, and the normal range is within a numerical range of the skin condition information in which it is determined that the skin condition of the user is maintained in a previous condition.

The determining of the excess of the normal range includes calculating a moving average of the skin condition information obtained by measuring the skin condition over N times (N is a positive integer number) before a present skin condition is measured, calculating a deviation based on one of the skin condition information obtained through the N-times measurements and the moving average calculated through each of the N-times measurements, and determining a maximum error by taking into account the deviation while the moving average is set to a standard of the normal range.

The method further includes searching for and providing prescription data in a case that the skin condition information presently measured exceeds the normal range, and the prescription data are determined depending on a numerical value of the skin condition information exceeding the normal range.

The method further includes receiving surrounding environmental condition data of a location where the user is located, and the searching for the prescription data includes searching for the prescription data corresponding to surrounding environmental condition by taking into account the surrounding environmental condition data, and the surrounding environmental condition data comprise at least one of a temperature, a humidity, an ultraviolet ray amount, a fine dust concentration, and an ozone concentration.

The calculating of the skin condition information includes correcting the skin condition information by taking into account the surrounding environmental condition data.

A skin condition analyzing method is combined with a hardware and to execute the above-mentioned method and stored in a medium.

According to the inventive concept, various effects are presented as described below.

First, the user may identify the skin condition of the desired skin area at the certain time point in real time. Accordingly, the user may check and perform the appropriate prescription corresponding to the skin condition, and thus the user may effectively maintain or improve the skin condition.

Second, the skin condition measuring device according to the exemplary embodiment of the inventive concept may check and manage not only the surface of the skin but also the inside of the skin (e.g., a dermal layer) using the alternating currents having different frequencies from each other.

Third, since the prescription data are searched based on the results obtained by measuring the skin moisture degree, the skin oiliness degree, and the skin sensitivity of the user, the prescription data appropriate to the skin condition of the user may be provided.

Fourth, the skin condition measuring device according to the exemplary embodiment of the inventive concept may obtain the skin image of the user under the same condition without being affected by the external light.

Fifth, the moving average of the skin condition information is set to the center of the error range, and the error range is set by the deviation of the moving average. Accordingly, the variation in the skin condition of the user may be measured. In addition, the errors caused by the external factors, such as the surrounding environmental condition or the skin condition, may be reduced, and thus the accuracy of the skin evaluation provided to the user may be improved.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a block diagram showing an inner configuration of a portable skin condition measuring device according to an exemplary embodiment of the inventive concept;

FIG. 2 is a view showing an appearance of a portable skin condition measuring device according to an exemplary embodiment of the inventive concept;

FIG. 3 is a flowchart showing a skin condition analyzing method according to an exemplary embodiment of the inventive concept;

FIG. 4 is a graph showing voltage data of input alternating current and voltage data of return alternating current, which have a specific frequency, according to an exemplary embodiment of the inventive concept; and

FIG. 5 is a view showing a degree of cell-permeability of an alternating current into a skin depending on different frequencies according to an exemplary embodiment of the inventive concept.

### DETAILED DESCRIPTION

The inventive concept and methods of accomplishing the same may be understood more readily by reference to the following detailed description of embodiments and the accompanying drawings. However, the inventive concept may be embodied in many different forms, and should not be construed as being limited to the embodiments set forth herein. Rather, these embodiments are provided so that this disclosure will be thorough and complete and will fully convey the concept of the invention to those skilled in the art, and the inventive concept will only be defined by the appended claims. Like numbers refer to like elements throughout.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The terminology used herein is for the purpose of describing particular embodiments only and is not intended to be limiting of the invention. As used herein, the singular forms are intended to include the plural forms as well, unless the context clearly indicates otherwise. It will be further understood that the terms “comprises” and/or “comprising”, when used in this specification, specify the presence of elements, but do not preclude the presence or addition of one or more other elements.

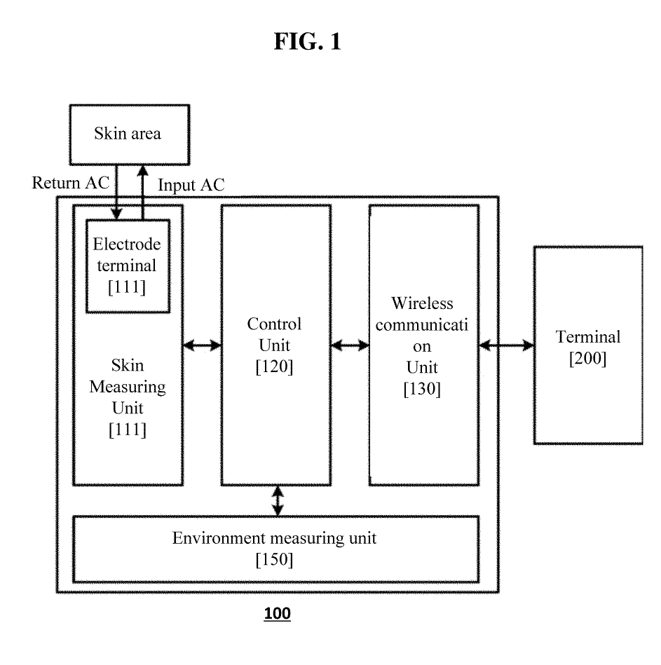

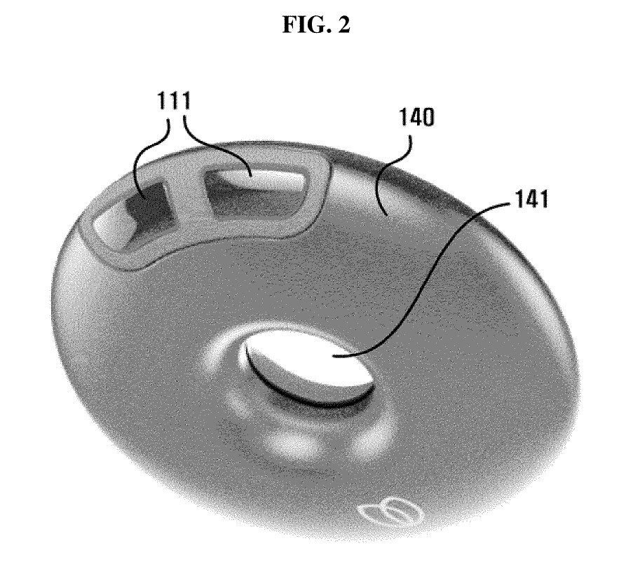

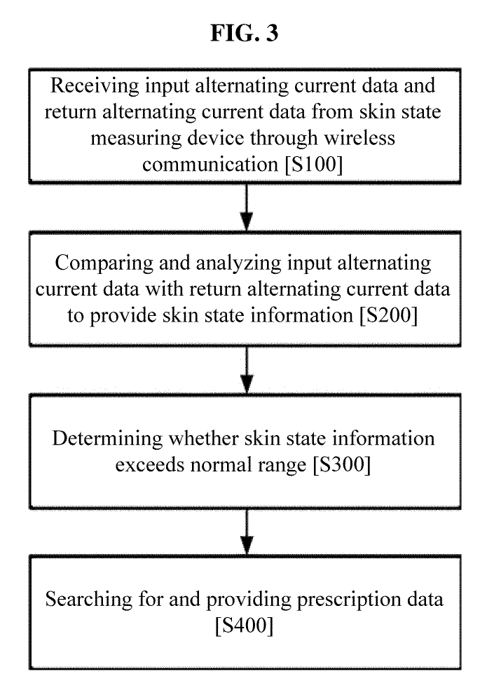

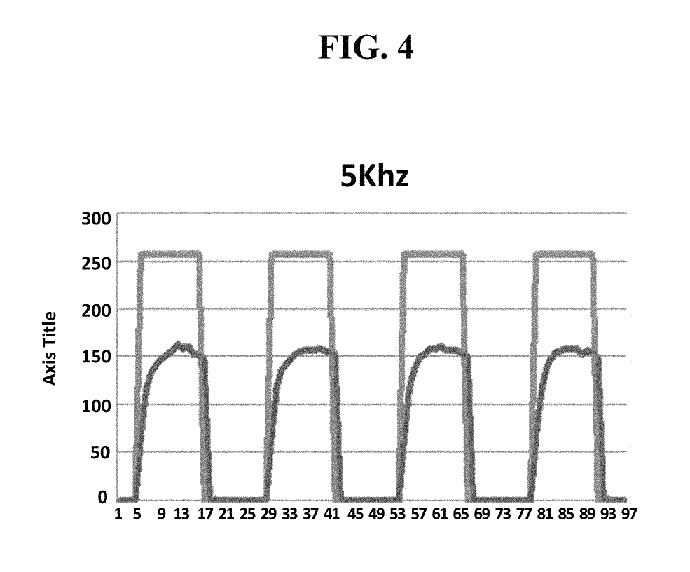

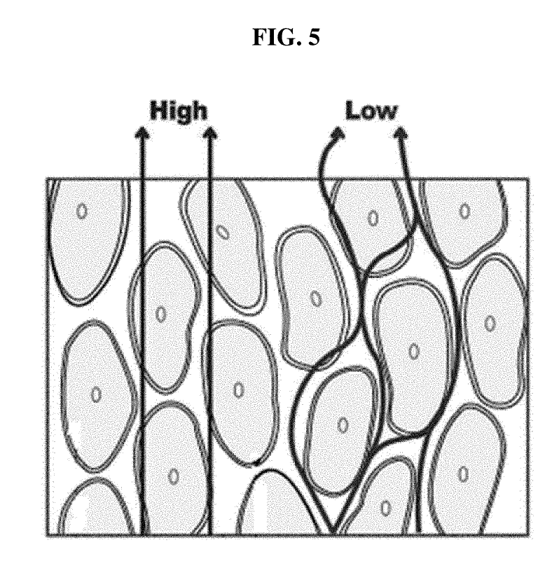

FIG. 1 is a block diagram showing an inner configuration of a portable skin condition measuring device according to an exemplary embodiment of the inventive concept. FIG. 2 is a view showing an appearance of a portable skin condition measuring device according to an exemplary embodiment of the inventive concept. FIG. 3 is a flowchart showing a skin condition analyzing method according to an exemplary embodiment of the inventive concept. FIG. 4 is a graph showing voltage data of input alternating current and voltage data of return alternating current, which have a specific frequency, according to an exemplary embodiment of the inventive concept. FIG. 5 is a view showing a degree of cell-permeability of an alternating current depending on different frequencies according to an exemplary embodiment of the inventive concept.

FIGS. 1 to 5 show a skin condition measuring device 100, a skin measuring unit 110, an electrode terminal 111, a control unit 120, a wireless communication unit 130, a housing 140, a recess part 141, an environment measuring unit 150, and a portable terminal 200.

Hereinafter, the portable skin condition measuring device 100 according to exemplary embodiments of the inventive concept will be described in detail with reference to accompanying drawings.

FIG. 1 is a block diagram showing an inner configuration of the portable skin condition measuring device 100 according to an exemplary embodiment of the inventive concept. FIG. 2 is a view showing an appearance of the portable skin condition measuring device 100 according to an exemplary embodiment of the inventive concept.

Referring to FIGS. 1 and 2, the portable skin condition measuring device 100 includes all or part of the skin measuring unit 110, the control unit 120, the wireless communication unit 130, the housing 140, and the environment measuring unit 150.

The housing 140 includes an installation space defined therein, and thus the skin measuring unit 110, the control unit 120, the wireless communication unit 130, and the environment measuring unit 150 may be installed in the housing 140. In addition, the housing 140 may include a hole defined therethrough to accommodate a power connector (e.g., an USB port or an external terminal connection) for the charge of the skin condition measuring device 100 with power. Further, in the case that the environment measuring unit 150 is installed inside the housing 140, the housing 140 may further include a ventilation opening through which air inflows, which is required to measure surrounding environmental conditions.

The housing 140 may have a shape that allows the user to hold the housing 140 conveniently. As an example, the housing 140 may have a disk shape allowing the user to hold the housing 140 conveniently and may be manufactured to have a size easy to hold with one hand.

In addition, the housing according to an exemplary embodiment of the inventive concept may have a ring shape. That is, the housing may be manufactured in the ring shape to be conveniently carried by connecting a string.

Further, the housing according to an exemplary embodiment of the inventive concept may include the recess part 141 that forms an inner space when making contact with a skin area. As an example, the recess part 141 may correspond to a part inwardly caved (or recessed) in the center of the disk shape as shown in FIG. 2. The recess part 141 may accommodate an imaging unit and a light supply unit therein, which are described later.

The skin measuring unit 110 applies an input alternating current having a predetermined frequency to the skin of the user and receives a return alternating current from the skin. The skin measuring unit 110 applies a specific alternating current to the skin through the electrode terminal 111. That is, the skin measuring unit 110 may include the electrode terminal 111 that applies the input alternating current to the skin area of the user and transmits the return alternating current to the skin measuring unit 110. The electrode terminal 111 may be installed on the housing to be exposed to the outside of the housing such that the electrode terminal 111 gets close to the skin of the user. The electrode terminal 111 may include an input electrode terminal to apply the input alternating current generated by the skin measuring unit 110 to the specific skin area and a receiving electrode terminal to receive the return alternating current returned from the skin area. The input electrode terminal and the receiving electrode terminal may be arranged at adjacent locations to be distinguished from each other.

The input electrode terminal and the receiving electrode terminal may be maintained in an open condition before making contact with the skin, and when the input electrode terminal and the receiving electrode terminal make contact with the skin, the input electrode terminal and the receiving electrode terminal may be connected to each other through the skin within a certain depth.

In addition, the input electrode terminal and the receiving electrode terminal may be arranged spaced apart from each other at a certain distance. As an example, when the input alternating current provided from the input electrode terminal passes through the skin by the certain distance, a voltage reduction and a variation in signal form may occur in a range in which the input alternating current may be analyzed.

The electrode terminal 111 may include a metal with high electrical conductivity. In addition, the electrode terminal 111 may be formed of a metal with low alteration, which does not affect the skin in use. For instance, the electrode terminal 111 may include the metal, such as gold, silver, chromium, etc.

The input alternating current corresponds to an alternating current having a specific frequency and applied to the skin of the user. The return alternating current corresponds to an alternating current returned to the skin measuring unit 110 after the input alternating current applied to the skin is returned from the certain depth of a skin layer.

As an example, the input alternating current may correspond to a rectangular wave having a specific maximum voltage, and the return alternating current may be obtained from the input alternating current in which a voltage is reduced at a specific rate depending on the skin condition or may have a shape of a specific output signal. In detail, the skin condition measuring device may generate the input alternating current by a pulse width modulation (PWM) method and input the input alternating current to the skin.

The input alternating current applied to the skin by the skin measuring unit 110 and the return alternating current corresponding to the input alternating current may be used to measure a skin moisture degree and a skin oiliness degree through comparisons with each other. The skin of the user may have a resistance and a capacitance due to the moisture degree and the oiliness degree in the skin. As an example, the voltage of the input alternating current may be reduced at a specific rate by the resistance according to the moisture degree of the skin when being input to the skin. In addition, the signal shape of the input alternating current may vary depending on the capacitance according to the oiliness degree of the skin.

In addition, the skin measuring unit 110 may apply input alternating currents having different frequencies from each other to specific skin areas of the user and acquire return alternating currents respectively corresponding to the frequencies. That is, the skin measuring unit 110 may sequentially apply plural input alternating currents having different frequencies from each other to the skin areas and receive the plural return alternating currents respectively corresponding to the input alternating currents. A depth of cell, to which the alternating current may reach after penetrating through the skin, becomes different depending on a magnitude of the frequency of the alternating current.

In detail, the return alternating currents may have different cell permeabilities from each other by each of the frequencies. The term “cell permeability” used herein means a degree to which a signal penetrates into and passes through the cell. As an example, when the frequency of the alternating current is low, the alternating current does not penetrate through the cell as shown in FIG. 5, and a rate at which the alternating current passes through boundaries between adjacent cells becomes high, and when the frequency of the alternating current is high, the alternating current passes through the cells while traveling as shown in FIG. 5. That is, the input alternating currents having the different frequencies move through the skin layer along different paths from each other due to different cell permeabilities and are returned. In a case that the skin moisture degree (or a skin moisture amount) is measured through the above-mentioned principle, since the cell permeability increases as the frequency of the input alternating current increases, an extracellular moisture degree may be measured using the low frequency, and a value including an intracellular moisture degree in addition to the extracellular moisture degree may be measured using the high frequency. Accordingly, the skin measuring unit 110 may combine the return alternating currents obtained by applying the input alternating currents having various frequencies, and thus the skin measuring unit 110 may calculate skin condition information (e.g., a degree of total skin moisture including the extracellular moisture degree and the intracellular moisture degree of the skin layer) with respect to the entire horny layer (stratum corneum) of the specific skin area (i.e., an area on which a skin measuring process is performed).

The control unit 120 controls an overall operation of the skin condition measuring device 100. The control unit 120 performs an information-processing function to process data obtained through the configurations (e.g., a user input unit receiving the user's operation, the skin measuring unit 110 receiving the return alternating current, the environment measuring unit 150 measuring the surrounding environmental conditions, etc.) in the skin condition measuring device 100. That is, the control unit 120 may perform a function for signal-processing the input alternating current and the return alternating current to generate conversion data. The conversion data may correspond to data obtained by converting the input alternating current and the return alternating current to digital signals to transfer the data to the external portable terminal 200 through a wireless communication.

In addition, the control unit 120 may perform a function for supplying a power to the configurations, e.g., the skin measuring unit 110, the wireless communication unit 130, etc., of the skin condition measuring device 100. The control unit 120 may perform a function for controlling an overall operation of the skin condition measuring device 100.

In addition, the control unit 120 may analyze the conversion data of the measured return alternating signal to calculate an analyzed result. That is, the control unit 120 obtains data, such as a difference in voltage between the input alternating current and the return alternating current, a deformation of the signal shape, etc., and analyzes the data to provide the analyzed result corresponding to the skin condition. As an example, the control unit 120 recognizes differences between the input alternating current and the return alternating current by comparing the input alternating current with the return alternating current and identifies the skin condition corresponding to the recognized difference based on stored databases.

In addition, the control unit 120 may calculate the analyzed result in consideration of data related the surrounding environmental condition measured by the environment measuring unit 150 described later. For instance, the control unit 120 may calculate and provide a risk of ultraviolet damage to the user's skin in consideration of a real-time UV amount at a location where the user is currently located.

In addition, the control unit 120 may include prescription data obtained by reflecting the skin condition of the user or the surrounding environmental condition, which are calculated in the analyzed result data. That is, the control unit 120 searches for the prescription data corresponding to the skin condition of the user and provides the prescription data with the skin condition information. The prescription data may include various information to suit the skin condition of the user. As an example, the prescription data may correspond to a recommendation of proper cosmetics, such as a sun protection product, a moisturizer, a functional cosmetics, etc., or a recommendation of foods, which help to improve or maintain the skin condition. In addition, the prescription data may correspond to dermatologist's comments corresponding to the skin condition of the user, which are stored in an external server or the portable terminal 200.

The control unit 120 may search for and provide the prescription data in database of the skin condition measuring device 100, in which the prescription data are stored. The database may be periodically updated through the wireless communication. In addition, the control unit 120 may receive the prescription data appropriate to the skin condition data or the surrounding environmental condition data after transmitting the skin condition data or the surrounding environmental condition data to the outside of the control unit 120 through the wireless communication.

The wireless communication unit 130 may perform a function for transmitting the conversion data generated by the control unit 120 to the portable terminal 200 of the user. In addition, the wireless communication unit 130 may perform a function for receiving setting information of the skin condition measuring device 100 from the portable terminal 200.

The wireless communication unit 130 may include one or more modules enabling the wireless communication between the skin condition measuring device 100 and a wireless communication system or between the skin condition measuring device 100 and a network at which the skin condition measuring device 100 is located (e.g., between the external portable terminal 200 having a wireless communication function and the skin condition measuring device 100). As an example, the wireless communication unit 130 may include a wireless internet module, a short range communication module, or the like.

The wireless internet module indicates a module for wireless internet access and is built in or mounted on the skin condition measuring device 100. As a wireless internet technology, a wireless LAN (WLAN), a Wireless Fidelity (Wi-Fi), a Wireless broadband (Wibro), a World interoperability for microwave access (Wimax), a High Speed Downlink Packet Access (HSDPA), a Long Term Evolution (LTE), or a Long Term Evolution-Advanced (LTE-A) may be used.

The short range communication module indicates a module for the short range communication. As the short range communication, a Bluetooth, a Bluetooth Low Energy (BLE), a Beacon, a Radio Frequency Identification (RFID), a Near Field Communication (NFC), an Infrared Data Association (IrDA), an Ultra Wideband (UWB), or a ZigBee may be used.

The BLE (Bluetooth Low Energy) indicates a Bluetooth communication method that enables faster communication by varying a configuration of packets to be different from that of a conventional classic Bluetooth and simplifying connection procedures. The BLE may be easily applied to items used in everyday life as a protocol improved to deliver information readout from sensors more quickly and easily. The BLE (Bluetooth 4.0) may communicate with devices within a range from about 5 m to about 70 m, and since the BLE consumes low power with little impact on battery life, the Bluetooth may be always in a turned-on state.

The NFC (Near Field Communication) module is a non-contact communication technology that is one of RFID technologies, and thus NFC module transmits and receives various wireless data in a distance of less than about 10 cm. The distance required for the NFC module is relatively short, the NFC module has superior security and low cost, and thus the NFC module is spotlighted as a next generation short range communication technology. In addition, since the NFC module may perform functions of reading and writing the data, the NFC module does not need a dongle (reader) required by the conventional RFID technology. Further, the NFC module is similar to the conventional short range communication technology, but the NFC module does not need to set up a connection between devices as does Bluetooth.

The wireless communication unit 130 may transmit the conversion data to the portable terminal 200 connected thereto in real time or may transmit the conversion data when being connected to the portable terminal 200 after temporarily storing the conversion data in a storing unit.

In addition, the wireless communication unit 130 may include a position measuring module. The position measuring module is a module to obtain a position of the skin condition measuring device 100, and a Global Positioning System (GPS) module is a representative example of the position measuring module. The position measuring module may check a position on which the user performs the skin condition measuring process or the environment condition measuring process is performed. Accordingly, the wireless communication unit 130 may transmit position information on the position, on which the user performs the skin condition measuring process or the environment condition measuring process is performed, to the external server or the portable terminal 200 such that the position information may be combined together with the skin condition information, and thus a prescription for improving or maintaining the skin condition of the user may be obtained by user's locations.

The environment measuring unit 150 may perform a function for measuring the surrounding environmental condition data of a location where the user is located. The surrounding environmental condition data may include a temperature, a humidity, an ultraviolet ray amount, a fine dust concentration, an ozone concentration, and the like. That is, the environment measuring unit 150 may include a temperature sensor, a humidity sensor, an ultraviolet ray sensor, an ozone sensor, and the like to measure the surrounding environmental condition data. In the case that the skin condition measuring device 100 makes contact with or is disposed on the skin area of the user to apply the alternating current to the user's skin, the environment measuring unit 150 may be located at a location suitable for measuring the surrounding environmental condition exerting influence on the skin area. As an example, the ultraviolet ray sensor is disposed on an opposite surface to a skin contact surface of the skin condition measuring device 100 to measure the amount of the ultraviolet ray irradiated onto the skin direction. The control unit 120 may generate the conversion data including the surrounding environmental condition data measured by the environment measuring unit 150.

In addition, the environment measuring unit 150 may accumulate data related to a time or a position of the surrounding environmental condition to which the skin of the user is exposed. The environment measuring unit 150 may store the surrounding environmental condition, to which the user is exposed, depending on the position information obtained by the position measuring module. Accordingly, it is possible to recognize the place where the user is mainly exposed to a certain surrounding environmental condition (e.g., the amount of the ultraviolet ray above a certain level). In addition, since the position measuring module may be used to measure the time, the accumulated time during which the user is exposed to the certain surrounding environmental condition may be checked.

The user input unit (not shown) may generate the input data to control the operation of the portable terminal 200 by the user. The user input unit may be implemented in a key pad, a dome switch, a touch pad (capacitive/resistive), a jog wheel, or a jog switch. In particular, in a case that the touch pad has a mutual layer structure with a display unit (not shown), this may be called a touch screen.

In addition, the exemplary embodiment of the inventive concept may further include an analyzed result output unit. In the case that the control unit calculates the analyzed result with respect to the skin condition based on the measured data, the analyzed result output unit performs a function of providing the analyzed result to the user. The analyzed result output unit may be implemented in a display method to provide the analyzed result visually. The analyzed result output unit may display a value or a graph of the specific skin condition by measuring the alternating signal in real time.

The analyzed result output unit may include at least one of a liquid crystal display (LCD) a thin film transistor-liquid crystal display (TFT-LCD), an organic light-emitting diode (OLED), a flexible display, a three-dimensional (3D) display, and an e-ink display.

Among them, some displays may be implemented in a transparent manner or in a light-transmitting manner These may be called a transparent display, and a transparent OLED (TOLED) is suggested as a representative example of the transparent display. A rear structure of the analyzed result output unit may be implemented in the light-transmitting manner. Due to the above-mentioned structure, the user may see objects disposed at a rear side of a body of the portable terminal through an area of the body of the portable terminal, which is occupied by the analyzed result output unit.

Two or more analyzed result output units may be provided to the skin condition measuring device depending on the implementation of the skin condition measuring device. As an example, a plurality of display units may be arranged on one surface to be spaced apart from each other or to be integrally formed with each other, or the display units may be disposed on different surfaces, respectively.

In the case that the analyzed result output unit has a mutual layer structure with the touch pad (hereinafter, referred to as the “touch screen”), the analyzed result output unit may be used not only as the output unit but also as an input unit. The analyzed result output unit may be provided on an outer surface of the housing. Particularly, the analyzed result output unit may be located at a location easy to check the analyzed result when the user holds the skin condition measuring device in his or her hand. As an example, in the case that the skin condition measuring device is manufactured to have a small size that fits inside the hand when held in the hand, the analyzed result output unit may be disposed on the same surface as the electrode terminal to allow the user to check the measured result right after being separated from the skin since it is difficult to check the analyzed result right after measuring the skin condition when the analyzed result output unit is disposed on the opposite surface to the surface on which the electrode terminal is disposed.

In addition, the exemplary embodiment of the inventive concept may further include the imaging unit and the light supply unit. The imaging unit may perform a function for taking a skin image of the specific skin area of the user. The obtained skin image may be used to analyze a skin tone, a pore size, a wrinkle degree, a condition of dead skin cells, and the like.

The light supply unit may perform a function for supplying a light with a specific intensity to the skin area when taking the skin image. The light supply unit supplies the light in many directions, and thus the light supply unit may provide the same brightness state regardless of the direction in which the user holds the skin condition measuring device 100.

In addition, the imaging unit and the light supply unit may be disposed in the recess part 141 and may take the skin image under the constant brightness state while the external light is blocked by the housing. That is, in the case that the imaging unit is disposed in the recess part 141 and the skin condition measuring device 100 is disposed to allow an entrance of the recess part 141 to make contact with the skin and take the skin image as shown in FIG. 2, the external light traveling toward the skin area that is to be photographed may be blocked by the housing. Then, the imaging unit may perform a function for taking the skin image while the light supply unit supplies the light with predetermined brightness in many directions. Accordingly, the skin condition measuring device 100 may obtain the skin tone measured under the same condition.

In addition, since the imaging unit is disposed in the recess part 141 recessed from an outermost surface of the housing by a certain depth, the imaging unit may take the skin image at a location spaced apart from the skin by a predetermined certain distance. Accordingly, the skin image may be obtained under a constant condition, and thus a variation in the skin condition of the user may be accurately obtained.

In addition, the exemplary embodiment of the inventive concept may further include a contact sensor. That is, the contact sensor is disposed adjacent to the electrode terminal 111 or the recess part 141 and senses the contact of the skin condition measuring device 100 with the skin area to request the control unit 120 to apply the input alternating current or to obtain the skin image by the imaging unit. Accordingly, the power is provided to the skin measuring unit 110 or the imaging unit and the light supply unit only when the skin condition measuring device 100 makes contact with the skin, and thus a power efficiency may be increased. In addition, errors caused by measuring the skin condition before the skin condition measuring device 100 properly approaches to the skin of the user may be reduced.

Hereinafter, a skin condition analyzing method and a skin condition analyzing program according to other exemplary embodiments of the inventive concept will be described with reference to accompanying drawings.

FIG. 3 is a flowchart showing the skin condition analyzing method according to an exemplary embodiment of the inventive concept.

Referring to FIG. 3, the skin condition analyzing method according to the exemplary embodiment of the inventive concept includes receiving the input alternating current data and the return alternating current data from the skin condition measuring device 100 through the wireless communication (S100) and comparing and analyzing the input alternating current data with the return alternating current data to provide the skin condition information (S200). The skin condition analyzing method according to the exemplary embodiment of the inventive concept will be sequentially described.

The skin condition analyzing method may be performed in the skin condition measuring device 100 or the portable terminal 200.

The skin condition measuring device 100 or the portable terminal 200 receives the input alternating current data and the return alternating current data (S100). That is, the control unit 120 of the skin condition measuring device 100 receives the input alternating current data and the return alternating current data from the skin measuring unit 110. The portable terminal 200 receives the input alternating current data and the return alternating current data from the skin condition measuring device 100 through the wireless communication. The input alternating current data and the return alternating current data may include an intensity of voltage, an intensity of current, a frequency of alternating current, and the like. In addition, the input alternating current data and the return alternating current data may correspond to graphs including waveforms of the voltage or the current.

The skin condition information is calculated by comparing and analyzing the input alternating current data with the return alternating current data (S200). The skin condition information may include the skin moisture degree, the skin oiliness degree, and a skin sensitivity.

The skin moisture degree measuring method according to an exemplary embodiment of the inventive concept may be performed by measuring an electrical conductivity based on a reduction amount of the output voltage to the input voltage and calculating the skin moisture degree. The moisture is required for current to flow through the skin. That is, the electrical conductivity increases as the moisture degree increases in the skin, and the electrical conductivity decreases as the moisture degree decreases in the skin. The portable terminal 200 or the control unit 120 of the skin condition measuring device 100 may measure the electrical conductivity of the skin using an impedance obtained by flowing the current having the specific frequency. The electrical conductivity means a degree to which a material may carry electric charges and corresponds to an inverse of resistivity (ρ). That is, the electrical conductivity is proportional to an inverse of impedance of a skin equivalent circuit corresponding to the skin of the user. The impedance causes a voltage drop in the skin equivalent circuit. That is, the voltage drop measured by comparing a voltage of the current (return alternating current) exiting from a medium after passing through the medium with the voltage of the input current (the input alternating current) is generated by an equivalent impedance of the skin equivalent circuit. Accordingly, the portable terminal 200 or the control unit 120 of the skin condition measuring device 100 may calculate the impedance of the skin equivalent circuit to measure the electrical conductivity. Since the electrical conductivity of the skin is proportional to the amount of the moisture, the portable terminal 200 or the control unit 120 of the skin condition measuring device 100 may calculate the moisture degree using the electrical conductivity.

The impedance of the skin equivalent circuit may be calculated based on a voltage drop amount of the current exiting from the medium after passing through the medium to the voltage of the current applied to the medium. A difference between the voltage (a blue graph), when the alternating current having the frequency of about 5 kHz is applied to the skin and the voltage (a red graph) that is reduced while passing through the skin and exiting from the skin is shown as in FIG. 4, and the impedance may be calculated based on a difference in area between the red graph and the blue graph. The portable terminal 200 or the control unit 120 of the skin condition measuring device 100 may calculate the electrical conductivity based on the calculated impedance (e.g., the impedance and the electrical conductivity are inversely related to each other). In the exemplary embodiment of the inventive concept, the portable terminal 200 or the control unit 120 of the skin condition measuring device 100 may calculate the skin impedance by measuring a resistance value according to the voltage drop. That is, since the skin impedance is proportional to the skin resistance value in the skin equivalent circuit, a size of the impedance may be calculated, which is proportional to the skin resistance value (or level) calculated based on the degree of the voltage drop caused by inputting the alternating current. The portable terminal 200 or the control unit 120 of the skin condition measuring device 100 may calculate a size of reactance based on the calculated size of the impedance and the calculated size of the resistance. In addition, the receiving of the alternating current data (S100) may receive the input alternating current data having different frequencies from each other and the return alternating current data corresponding to the input alternating current data, and the providing of the skin condition information (S200) may calculate accumulated skin condition information by reflecting the skin condition information measured by each of the frequencies. The cell permeability may be changed depending on the frequency of the alternating current. The term “cell permeability” used herein means the degree to which the signal penetrates into and passes through the cell. As an example, when the frequency of the alternating current is low, the alternating current does not penetrate through the cell as shown in FIG. 5, and the rate at which the alternating current passes through between the cell and adjacent cells becomes high, and when the frequency of the alternating current is high, the alternating current proceeds after passing through the cells as shown in FIG. 5. Accordingly, in a case that the skin moisture degree (or a skin moisture amount) is measured, since the cell permeability increases as the frequency of the input alternating current increases, the extracellular moisture degree may be measured using the low frequency, and the value including the intracellular water content in addition to the extracellular water content may be measured using the high frequency.

Accordingly, the portable terminal 200 or the control unit 120 of the skin condition measuring device 100 may calculate a comprehensive skin moisture degree on which the extracellular moisture degree and intracellular moisture degree of the specific skin area are reflected through the combination of the input alternating current data having the various frequencies and the return alternating current data. The portable terminal 200 or the control unit 120 of the skin condition measuring device 100 may improve an accuracy of the skin moisture degree in the skin area on which the skin condition measurement is performed by taking into account the voltage drop amount (i.e., the difference in area between the input alternating current graph and the return alternating current graph). That is, the portable terminal 200 or the control unit 120 of the skin condition measuring device 100 may calculate the accumulated skin condition information (e.g., the skin moisture degree of the entire horny layer) with the skin condition information (e.g., the skin moisture degree) with respect to the entire horny layer.

In addition, the providing of the skin condition information (S200) may calculate the skin oiliness degree based on the calculated skin moisture degree. The skin oiliness degree and the skin moisture degree may have a corresponding relationship with each other according to the skin condition of the user. Accordingly, the portable terminal 200 or the control unit 120 of the skin condition measuring device 100 may calculate the skin oiliness degree based on the calculated skin moisture degree. In addition, the portable terminal 200 or the control unit 120 of the skin condition measuring device 100 may calculate the skin oiliness degree more accurately by taking into account other skin condition information of the user.

Further, the calculating of the skin oiliness degree may calculate the skin oiliness degree using the skin moisture degree through various manners. As an example, the portable terminal 200 or the control unit 120 of the skin condition measuring device 100 may calculate the skin oiliness degree based on a rate of change in the measured skin moisture degree. The oil of the skin prevents the moisture of the skin from evaporating. Accordingly, the portable terminal 200 or the control unit 120 of the skin condition measuring device 100 may calculate the skin oiliness degree by calculating a variation in amount of the skin moisture during a certain time period. In addition, the portable terminal 200 or the control unit 120 of the skin condition measuring device 100 may more accurately calculate data in consideration of the surrounding environmental condition between a first measuring time point and a second measuring time point. That is, since various factors, such as a temperature, a humidity, etc., at a location at which the user is located exert an influence on evaporation of the moisture, the skin oiliness degree may be calculated by reflecting the received surrounding environmental condition data.

In addition, the method may further include providing questionnaire information when the skin of the user is first measured and calculating an initial oiliness condition based on a feedback of the questionnaire information. Information, e.g., a skin type of the user, is required to allow the portable terminal 200 or the control unit 120 of the skin condition measuring device 100 to calculate the skin oiliness degree based on the calculated skin moisture degree. Accordingly, the portable terminal 200 or the control unit 120 of the skin condition measuring device 100 may identify the skin type by providing the questionnaire information and receiving the feedback with respect to the questionnaire information. Accordingly, the oiliness condition (i.e., the skin oiliness degree) at the beginning of the measurement of the user may be calculated. In addition, the exemplary embodiment of the inventive concept may further include determining whether the skin condition information exceeds a normal range (S300). The normal range may be a numerical range of the skin condition information, which is determined that the skin condition of the user is maintained in a previous condition. Errors may occur in the skin condition depending on the surrounding environmental condition (e.g., the temperature, the humidity, etc.) or the skin condition (e.g., whether sweating). Accordingly, when a variation exceeding a certain range corresponding to the normal range occurs in the skin condition, the portable terminal 200 or the control unit 120 of the skin condition measuring device 100 may recognize occurrence of the variation in the skin condition.

The exemplary embodiment of the inventive concept may set the normal range with reference to a moving average (MA). Since the errors might be generated due to external factors when the skin condition information is previously measured, setting the previously-measured skin condition information to a center of the normal range or a reference may decrease an accuracy with respect to the variation of the skin condition that is presently measured. Accordingly, the portable terminal 200 or the control unit 120 of the skin condition measuring device 100 may accumulate the skin condition information (e.g., the skin moisture degree) obtained by measuring the skin over N times to calculate the moving average. When the calculated moving average is set as a standard of error range, errors generated during N-times measurement of the skin condition are reflected to calculate the average. Accordingly, the influence of previous measurement errors of the skin condition information in the setting of the error range may be reduced. The N times may be the total number of the measurement times from the initial measurement and may be the number of measurement times after the measurement that determines the occurrence of the variation in the skin condition.

In addition, according to the exemplary embodiment of the inventive concept, the portable terminal 200 or the control unit 120 of the skin condition measuring device 100 may calculate a deviation (δ) of the moving average to set the normal range, which has the moving average as its center. That is, in a case that a variability of the value obtained by measuring the skin condition over N times is small, the variation amount, which determines the occurrence of the variation in the skin condition, needs to be set small, but in a case that the variability of the value obtained by measuring the skin condition over N times is large, the variation amount, which determines the occurrence of the variation in the skin condition, needs to be set large. To this end, the portable terminal 200 or the control unit 120 of the skin condition measuring device 100 may calculate the deviation (δ) of the moving average with respect to the skin condition information obtained by measuring the skin condition over N times and set a possible error range of [MA+Kδ, MA-Kδ] (K corresponds to a constant used to set a proper range). When it is determined that the measured skin condition information is out of the normal range, the portable terminal 200 or the control unit 120 of the skin condition measuring device 100 may determine that the prescription data need to be provided.

In addition, the skin condition analyzing method may further include searching for and providing the prescription data corresponding to the skin condition of the user (S400). That is, the portable terminal 200 or the control unit 120 of the skin condition measuring device 100 may search for appropriate prescription data based on the measured skin condition of the user and provide the searched prescription data.

The prescription data may be determined depending on how much the skin condition information exceeds the normal range. As an example, in the case that the skin condition information of the user exceeds by far the normal range, the portable terminal 200 or the control unit 120 of the skin condition measuring device 100 may determine that a sudden variation occurs in the skin condition, and thus the portable terminal 200 or the control unit 120 of the skin condition measuring device 100 may search for and provide the prescription data for emergency or powerful solutions.

The prescription data may include various information corresponding to the skin condition of the user. As an example, the prescription data may correspond to the recommendation of cosmetics, such as the sun protection product, the moisturizer, the functional cosmetics, etc., or the recommendation of foods, which helps to improve or maintain the skin condition.

In addition, the prescription data may correspond to dermatologist's comments corresponding to the skin condition of the user, which are stored in a database of an external server, the portable terminal 200, or the storing unit in the skin condition measuring device 100.

Further, the skin condition analyzing method may further include receiving the surrounding environmental condition data of the location where the user is located. The surrounding environmental condition data may include the temperature, the humidity, the ultraviolet ray amount, the fine dust concentration, the ozone concentration, and the like. The portable terminal 200 or the control unit 120 of the skin condition measuring device 100 may receive the surrounding environmental condition data obtained by the environment measuring unit 150 of the skin condition measuring device 100 and search for and receive the surrounding environmental condition data corresponding to the location of the user from the external server (e.g., a weather station server). The surrounding environmental condition data may be reflected to calculate the prescription data. That is, the searching for the prescription data (S400) may search for the prescription data appropriate to the surrounding environmental condition by taking into account the surrounding environmental condition data.

The portable terminal 200 or the control unit 120 of the skin condition measuring device 100 may reflect the surrounding environmental condition data not only to calculate the prescription data but also to calculate the skin condition information based on the return alternating current data with respect to the input alternating current data. That is, since the errors may occur in the skin condition information according to the surrounding environmental condition, the errors may be corrected by taking into account the surrounding environmental condition data.

The skin condition analyzing method according to the exemplary embodiment of the inventive concept may be implemented in a program (or an application) and stored in a medium to be executed after being combined with the portable skin condition measuring device 100 or the portable terminal 200 that is a hardware device.

The portable terminal 200 may include various devices that perform computational processing and provide the processed result to the user. As an example, the portable terminal 200 may be, but not limited to, a desktop personal computer, a notebook computer, a smartphone, a tablet personal computer, a cellular phone, a personal communication service (PCS) phone, a synchronous/asynchronous international mobile telecommunication-2000 (IMT-2000), a palm personal computer, a personal digital assistant (PDA), and the like.

The above-mentioned program may include codes coded in a computer language, such as C, C++, JAVA, a machine language, or the like, which are readable by a processor (CPU) of the computer through a device interface of the computer. These codes may include functional codes related to a mathematical function in which the functions required to execute the methods described above are defined and may include control codes related to execution procedures, which are required for the processor of the computer to execute the functions described above according to predetermined procedures. In addition, these codes may further include additional information required for the processor of the computer to execute the functions described above or memory reference-related codes as to where the media needs to be referenced (address) in an internal or external memory of the computer. In addition, in the case where the processor of the computer needs to communicate with any other remote computer or server to execute the functions described above, the codes may further include communication-related codes with respect to how the processor of the computer communicates with any other remote computer or server using a communication module of the computer, what kind of information or media is to be transmitted and received in the communication, and the like.

The recording medium indicates a medium that semi-permanently stores the data and that is readable by machines other than a medium that stores the data for a short time, such as a register, a cache, a memory, etc. In detail, examples of the recording medium in which the program is recorded include ROM, RAM, CD-ROM, a magnetic tape, a floppy disc, an optical media storage device, and the like, but it should not be limited thereto or thereby. That is, the program may be recorded in various recording media of various servers to which the computer may access or in various recording media of the computer of the user. In addition, the recording medium may be distributed over a network coupled computer system, so that the computer-readable code may be stored and executed in a distributed fashion.

According to the inventive concept, various effects are presented as described below.

First, the user may identify the skin condition of the desired skin area at the certain time point in real time. Accordingly, the user may check and perform the appropriate prescription corresponding to the skin condition, and thus the user may effectively maintain or improve the skin condition.

Second, the skin condition measuring device according to the exemplary embodiment of the inventive concept may check and manage not only the surface of the skin but also the inside of the skin (e.g., a dermal layer) using the alternating currents having different frequencies from each other.

Third, since the prescription data are searched based on the results obtained by measuring the skin moisture degree, the skin oiliness degree, and the skin sensitivity of the user, the prescription data appropriate to the skin condition of the user may be provided.

Fourth, the skin condition measuring device according to the exemplary embodiment of the inventive concept may obtain the skin image of the user under the same condition without being affected by the external light.

Fifth, the moving average of the skin condition information is set to the center of the error range, and the error range is set by the deviation of the moving average. Accordingly, the variation in the skin condition of the user may be measured. In addition, the errors caused by the external factors, such as the surrounding environmental condition or the skin condition, may be reduced, and thus the accuracy of the skin evaluation provided to the user may be improved.

While the inventive concept has been described with reference to embodiments, it will be apparent to those skilled in the art that various changes and modifications may be made without departing from the spirit and scope of the inventive concept. Therefore, it should be understood that the above embodiments are not limiting, but illustrative.

What is claimed is:

1. A method for analyzing a skin condition of a user with a skin condition measuring device or a portable terminal connected to the skin condition measuring device, comprising:
receiving input alternating current data and return alternating current data; and
comparing the input alternating current data with the return alternating current data to calculate skin condition information.

2. The method of claim 1, wherein, when the input alternating current data and the return alternating current data respectively correspond to voltage data and the skin condition information is a skin moisture degree, the calculating of the skin condition information comprises:
comparing the voltage data of the input alternating current data with the voltage data of the return alternating current data to calculate voltage drop data;
calculating an equivalent impedance based on the calculated voltage drop data;
calculating an electrical conductivity based on the equivalent impedance; and
calculating the skin moisture degree corresponding to the electrical conductivity.

3. The method of claim 1, wherein the receiving of the input alternating current data and the return alternating current data comprises receiving the input alternating current data having a plurality of different frequencies and the return alternating current data corresponding to the input alternating current data, the calculating of the skin condition information reflects the skin condition information by frequency, which is calculated depending on each of the frequencies, to calculate accumulated skin condition information, and the skin condition information by frequency corresponds to skin condition information obtained by alternating currents that have different frequencies from each other and move along different paths from each other in a skin layer due to a cell permeability by frequency.

4. The method of claim 2, further comprising calculating a skin oiliness degree corresponding to the calculated skin moisture degree.

5. The method of claim 1, further comprising determining whether the skin condition information exceeds a normal range, wherein the normal range is within a numerical range of the skin condition information in which it is determined that the skin condition of the user is maintained in a previous condition.

6. The method of claim 5, wherein the determining of the excess of the normal range comprises:
calculating a moving average of the skin condition information obtained by measuring the skin condition over N times (N is a positive integer number) before a present skin condition is measured;
calculating a deviation based on one of the skin condition information obtained through the N-times measurements and the moving average calculated through each of the N-times measurements; and
determining a maximum error by taking into account the deviation while the moving average is set to a standard of the normal range.

7. The method of claim 5, further comprising searching for and providing prescription data in a case that the skin condition information presently measured exceeds the normal range, wherein the prescription data are determined depending on a numerical value of the skin condition information exceeding the normal range.

8. The method of claim 7, further comprising receiving surrounding environmental condition data of a location where the user is located, wherein the searching for the prescription data comprises searching for the prescription data corresponding to surrounding environmental condition by taking into account the surrounding environmental condition data, and the surrounding environmental condition data comprise at least one of a temperature, a humidity, an ultraviolet ray amount, a fine dust concentration, and an ozone concentration.

9. The method of claim 8, wherein the calculating of the skin condition information comprises correcting the skin condition information by taking into account the surrounding environmental condition data.

10. A program for measuring a skin condition, which is being combined with a portable terminal that is hardware and being stored in a medium to execute a method of claim 1.
